(19) United States
(12) Patent Application Publication
(10) Pub. No.: US 2005/0181059 A1
(43) Pub. Date: Aug. 18, 2005

(54) Nanoparticulate therapeutic biologically active agents

(75) Inventors: Jules S. Jacob, Taunton, MA (US); Yong S. Jong, Providence, RI (US); Danielle T. Abramson, Boston, MA (US); Edith Mathiowitz, Brookline, MA (US); Camilla A. Santos, Newport, RI (US); Michael J. Bassett, Rumford, RI (US); Stacia Furtardo, Groton, CT (US)

Correspondence Address: PATREA L. PABST;PABST PATENT GROUP LLP, 400 COLONY SQUARE, SUITE 1200, ATLANTA, GA, 30361, US

(73) Assignees: Spherics, Inc.; Brown University Research Foundation

(21) Appl. No.: 10/954,423

(22) Filed: Sep. 30, 2004

Related U.S. Application Data
(60) Provisional application No. 60/507,413, filed on Sep. 30, 2003.

(51) Int. Cl. A61K 38/28; A61K 48/00; A61K 9/14

(52) U.S. Cl. 424/489; 514/3; 514/44

(57) ABSTRACT

Compositions containing particles of biologically active agents with sizes in the micron and submicron range and methods for making and using such particles are described herein. In the preferred embodiment the biologically active agents are peptides, proteins, nucleic acid molecules, or hydrophilic synthetic molecules. The particles have a size ranging from an average diameter of about 100 nm to about 2000 nm, preferably about 200 nm to 600 nm. Optionally the biologically active agents contain a polymeric coating. The particles are formed by adding a biologically active agent to an aqueous solution, mixing a nonsolvent that is miscible with water with the aqueous solution, and precipitating particles of the biologically active agents out of the nonsolvent: aqueous solution combination. The nonsolvent is typically a C1 to C6 alcohol, preferably a C2 to a C5 alcohol. In the preferred embodiment, the nonsolvent is tert-butyl alcohol.

### CROSS-REFERENCE TO RELATED APPLICATIONS

This application claims priority to U.S. provisional application Ser. No. 60/507,413, entitled “Nanoparticulate Therapeutic Biologically active agents” to Jules S. Jacob, Yong S. Jong, Danielle T. Abramson, Edith Mathiowitz, Camilla A. Santos, Michael J. Bassett, and Stacia Furtado, filed Sep. 30, 2003.

### FIELD OF THE INVENTION

The present invention relates to biologically active agents in the form of nanoparticles and microparticles.

### BACKGROUND OF THE INVENTION

Drug delivery of large biologically active agents, such as proteins, RNA and DNA, is often restricted to parenteral applications due to the size of the particles. Smaller sized biologically active agents, such as in the micron or submicron range, allows for the biologically active agents to be delivered using non-parenteral methods.

The micronization of proteins and drugs to form solid particles suitable for microencapsulation (e.g., particles having a size less than about 10 μm) has been achieved using a variety of approaches including milling, spray-drying, spray freeze-drying, and supercritical anti-solvent (SAS) precipitation techniques. While proteins are generally more stable in a lyophilized (dry) state than a hydrated state, it is often difficult to produce dry micronized (less than 20 μm) protein particulates. The particle size is critical to drug release kinetics of matrix type devices.

Various milling techniques to reduce the particle size of biologically active agents are known (see e.g. U.S. Pat. No. 5,952,008 to Backstrom et al.; U.S. Pat. No. 5,354,562 to Platz et al.; U.S. Pat. No. 5,747,002 to Clark et al.; and U.S. Pat. No. 4,151,273 to Riegelman et al.). Methods employing supercritical conditions also are well known (see e.g. U.S. Pat. No. 5,043,280 to Fischer et al.; U.S. Pat. No. 5,851,453 to Hanna et al.; U.S. Pat. Nos. 5,833,891 and 5,874,029 to Subramaniam et al.; and U.S. Pat. No. 5,639,441 to Sievers et al.).

Spray drying methods also are well known in the art (see e.g. U.S. Pat. No. 5,700,471 to End et al.; U.S. Pat. No. 5,855,913 to Hanes et al.; U.S. Pat. No. 5,874,064 to Edwards et al.; and Komblum, J. Pharm. Sci. 58(1):125-27 (1969)). Precipitation techniques that can reduce the size of the particles of biologically active agent are also known (see e.g. U.S. Pat. No. 5,776,495 to Duclos et al.; U.S. Pat. No. 4,332,721 to Bernini et al.; U.S. Pat. No. 5,800,834 to Spireas et al.; U.S. Pat. No. 5,780,062 to Frank et al.; and U.S. Pat. No. 5,817,343 to Burke). Sonication is another technique employed to micronize particles (see e.g. U.S. Pat. No. 4,384,975 to Fong et al. and Tracy, Biotechnol. Prog, 14:108-15 (1998)).

However, some of these methods are not desirable for micronizing certain types of agents, such as proteins. For example, exposure to high temperatures or an aqueous/organic solvent interface is known to be detrimental to protein stability leading to denaturation. It would be advantageous to provide dry, micronized particles of biologically active agents, and a method of making such particles which substantially avoids or minimizes denaturation of the biologically active agents. It would also be advantageous to provide dry micronized particles having a small, uniform size.

Methods for encapsulating and micronizing particles of agent have been described in U.S. Pat. Nos. 6,677,869; 6,235,224; and 6,143,211 to Mathiowitz et al. and Mathiowitz et al., Nature 386: 410 (1997). The patents and publication describe a method of encapsulating drugs in micron and sub-micron polymeric microspheres. In this method, called Phase Inversion Nanoencapsulation (“PIN”), a polymer is dissolved in a solvent and the drug or other material to be encapsulated is dissolved or suspended in the polymer solution. The resulting solution or suspension is rapidly diluted with a solution that is a non-solvent for the polymer, and preferably for the drug or agent. The non-solvent is selected to be sufficiently miscible with the solvent so that a single-phase solution that is a non-solvent for the polymer is formed after the dilution. The spontaneous mixing of the two solutions occurs rapidly and with a small characteristic scale of mixing. As a result, the polymer precipitates to form particles with a very small diameter, typically in the range of tens to hundreds of nanometers, or in some cases up to several microns in diameter. These particles are generally uniform in size. The drug or agent is encapsulated in the nanospheres. Upon administration to a patient, or other application, the drug or agent is released from the nanospheres by diffusion, degradation of the polymer, or a combination of these effects.

In some situations, the presence of an encapsulating polymer may be unnecessary, or even inhibiting, in the delivery of a drug. Methods for micronizing biologically active agents with low aqueous solubility, such as taxanes, have been described in PCT/US03/34575 to Spherics Inc. However, different methods may be useful for other biologically active agents, such as peptides, proteins, nucleic acid molecules, and hydrophilic synthetic molecules.

Therefore, it is an object of the invention to provide a method for producing particles of biologically active agents in the micron and submicron size that preserves the native structure or activity of the biologically active agents.

It is a further object of the invention to provide particles of biologically active agentsin the micron and submicron size range.

It is a further objective of the invention to produce particles of biologically active agents in the micron and submicron size range that can be used in drug compositions which are administered by conventional routes of administration, particularly via the oral route.

### SUMMARY OF THE INVENTION

Compositions containing particles of biologically active agents with sizes in the micron and submicron range and methods for making and using such particles are described herein. In the preferred embodiment the biologically active agents are peptides, proteins, nucleic acid molecules, or hydrophilic synthetic molecules. The particles have a size ranging from an average diameter of about 100 nm to about 2000 nm, preferably about 200 nm to 600 nm. Optionally the biologically active agents contain a polymeric coating. The particles are formed by adding a biologically active agent to an aqueous solution, mixing a nonsolvent that is miscible with water with the aqueous solution, and precipitating particles of the biologically active agents out of the nonsolvent: aqueous solution combination. The nonsolvent is typically a C 1 to C6 alcohol, preferably a C2 to a C5 alcohol. In the preferred embodiment, the nonsolvent is tert-butyl alcohol.

### DETAILED DESCRIPTION OF THE INVENTION

I. Compositions

The compositions contain small particles of biologically active agents. As generally used herein, “biologically active agents” includes polymeric molecules, such as proteins, peptides, and nucleic acids (RNA and DNA), and synthetic or semisynthetic analogs thereof, that are used for therapy, diagnosis, prophylaxis, or immunization. The particles are a population of nanoparticles in which the average diameter is between 100 nm and 2000 nm. The particles of agent are generally stable, and do not aggregate irreversibly.

In the preferred embodiment the particles have diameters in the submicron range, such as from 200 nm to 600 nm. The particles of drug may be present in the composition with or without a coating.

Optionally, the particles are encapsulated in one or more polymers. A variety of excipients and additives may be present, especially additives for preventing particle aggregation, and additives to preserve biological activity.

A. Biologically Active Agents

Many different biologically active agents may be formed into small particles by the methods described herein. Biologically active agents include synthetic and natural proteins (including enzymes, peptide-hormones, receptors, growth factors, antibodies, signalling molecules), and synthetic and natural nucleic acids (including RNA, DNA, anti-sense RNA, triplex DNA, inhibitory RNA (RNAi), and oligonucleotides), and biologically active portions thereof.

Suitable biologically active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”) Nucleic acids with lengths above about 10 bp are typically used in the present method. More typically, useful lengths of nucleic acids for probing or therapeutic use will be in the range from about 20 bp (probes; inhibitory RNAs, etc.) to tens of thousands of bp for genes and vectors. The biologically active agents may also be hydrophilic molecules, preferably having a low molecular weight.

After the biologically active agents are micronized to form small particles, they retain a significant and therapeutically useful level of recoverable biologic activity. Preferably, the preparation retains at least 50% of it original biological activity, and more preferably the preparation retains 60-90% of its original biological activity, based on the weight of biologically active agent in the sample compared to an equal weight of the original biologically active agent. In the most preferred embodiment, the preparation retains greater than 90% of its original biological activity. The biological activity may be any type of biological activity, including hormonal, enzymatic, binding, recognition, stimulatory, inhibitory, transformation, or recombination activities, gene silencing, gene probing, gene expression, or behaving as a ligand or cofactor.

The method of determining biological activity varies with the particular biologically active agent, and can be found in the scientific literature describing the biological activity of the biologically active agent, or in literature associated with the biologically active agent's approval as a therapeutic substance. When available, bioassay, i.e. observation of the level of the material in the blood or other tissue, or observation of the effect of the biologically active agent (e.g., lowering of blood sugar by insulin) is the preferred route of assay. Methods assessing the absence of denaturation in an active biologically active agent may include analytical methods sensitive to aggregation of molecules or to breakage of molecular structure. Many such methods are known and are potentially suitable, of which the most common are chromatography, particularly sieving by molecular weight, and gel electrophoresis, either in the native state or specifically denatured, for example by detergents or changes in pH. Mass spectroscopy, ultracentrifugation, optical and magnetic resonance spectroscopy, electron and atomic probe microscopy and other physical methods may also be useful.

B. Size of Particles

The particles as prepared have an average diameter ranging from 100 to 2000 nm. As generally used herein “average diameter” refers to a volume-average diameter and may be determined using scanning electron microscope (SEM) analysis. Typically, the particles are less than about 1 micron in diameter, and often in the range of about 200 nm to about 600 μm. The particle dispersion is relatively narrow, without normally being monodisperse. Typically greater than 90%, preferably more than 95%, more preferably more than 99%, of the particles have a diameter of less than 1 micron.

C Polymers

The particles may be initially provided in a state in which they do not have polymer coatings, although a fraction of polymer may be included in the composition as a stabilizer or other additive. However some applications may require that the particles contain a coating to accomplish delivery of drug to the appropriate site.

Non-biodegradable or biodegradable polymers may be used to encapsulate the biologically active agents. In the preferred embodiment, the particles are encapsulated in a biodegradable polymer. Non-erodible polymers may be used for oral administration. In general, synthetic polymers are preferred, although natural polymers may be used and have equivalent or even better properties, especially some of the natural biopolymers which degrade by hydrolysis, such as polyhydroxybutyrate. The coating may be formed during the formation of the particles, or may be applied in a later operation by the same or other methods.

Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the matrix can be adjusted during the production by using polymers such as polylactidecoglycolide copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof.

Bioadhesive polymers of particular interest for use in targeting of mucosal surfaces, as in the gastrointestinal tract, include polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

D. Carriers, Excipients and Stabilizers

The compositions may include a physiologically or pharmaceutically acceptable carrier, excipient, or stabilizer mixed with the micronized drug particles. The term “pharmaceutically acceptable” means a non-toxic material that does not interfere with the effectiveness of the biological activity of the active ingredients. The term “pharmaceutically-acceptable carrier” means one or more compatible solid or liquid fillers, dilutants or encapsulating substances which are suitable for administration to a human or other vertebrate animal. The term “carrier” refers to an organic or inorganic ingredient, natural or synthetic, with which the active ingredient is combined to facilitate the application.

II. Methods for Making Micron and Submicron Particles

The biomaterials are micronized to form solid particles suitable for delivery via any of a variety of routes, including among others oral delivery or inhalation. The preferred size (diameter) of a particle for such applications is less than about 10 microns, preferably less than 1 micron. The method of micronization involves mixing or spraying an aqueous solution of the biologically active agents into a selected nonsolvent that is miscible with water, which is typically a C 1 to C6 aliphatic alcohol or a mixture of such alcohols. One of the advantages of the method is that the microparticles of drug can be formed without any polymer coating. This makes them suitable for incorporation in a variety of delivery systems.

First, the biologically active agents are dissolved in an aqueous solution, which optionally includes one or more stabilizers, surfactants and/or other excipients. In this step, the aqueous solution is optionally brought to a state just short of precipitation of the biologically active agent. For example, a solution of biologically active agent can be titrated with a reagent until the beginning of precipitation is visible as opalescence in the solution. At that point, the solution is back-titrated with an opposing reagent to regain transparency. However, other biologically active agents, including some proteins and many peptides and nucleic acids, do not require this adjustment.

Next, in one embodiment, the aqueous biologically active agent solution, optionally with stabilizers and/or surfactants or other excipients is sprayed into a liquid nonsolvent, which is soluble with water. Particles of biologically active agents are formed as the water is extracted from the drops of spray into the nonsolvent. Alternatively, in another embodiment, the aqueous biologically active agent solution is simply mixed with an excess of nonsolvent, for example at least a 5-fold excess in volume, or a higher dilution ratio such as a 10-fold, or a 15 to 50-fold excess in volume.

The preferred amount of non-solvent is the smallest amount that will reliably form microparticles of the desired size; this is readily determined by experiment for each particular combination of biologically active agent and nonsolvent. Because the aqueous solution and nonsolvent are miscible, there is no surface tension to separate droplets. At most, mild stirring is needed to mix the solutions. The resulting particles for either method, spraying or mixing, often have an average diameter of less than 1 micron, typically in the range of about 200 to 600 nm. The primary criteria in preparation of these particles is the preservation of bioactivity for the agent.

Third, the particles formed by these methods are collected, and dried or vacuum dried as required. The drying methods used are conventional and include, among others, filtration, centrifugation, and freeze-drying.

A. Non-Solvents

The range of useful non-solvents is limited. When most organic solvents are used, gross denaturation is observed, and many biologically active agents form irreversible clumps. This is seen even with fairly polar organic solvents, such as acetone or ethyl acetate. The non-solvent must be miscible in water. A useful non-solvent for the micronization process is able to absorb water in the range of 2-100% w/w.

The preferred non-solvents of the method are the lower alcohols, i.e. C 1 to C6 alcohols. The most preferred non-solvent is tert-butyl alcohol, also identified as 2-methyl-propan-2-ol, and herein referred to as “t-butanol” or “tBA”. When t-butanol is used, the bioactivity agent is preserved and particles with small diameters are formed. Other suitable non-solvents include methanol, ethanol, propanol, isopropanol, other butanols (e.g. 1-butanol and 2-butanol), and pentanols, (e.g. 1-pentanol, 2-pentanol and 3-pentanol, 3-methyl-1-butanol (isopentanol), and tert-amyl alcohol). These are generically described as C1-C5 alcohols. Some of the C6 alcohols may also be useful. C1, C2 and C3alcohols, i.e. methanol, ethanol, and the propanols, typically produce small particles but often may require stabilizing or disaggregating agents to prevent particle aggregation during particle collection. Given prevention or reversal of aggregation, C 1, C2 and C3 alcohols are suitable, and have primary particles sizes similar to those found with t-butanol. The propanols, ethanol, and methanol, when the particle size is right and aggregation is controlled, are preferred for minimizing the cost of the method.

The butanols (other than tBA) and the pentanols are not miscible with water in all proportions, but all will absorb significant quantities of water. A useful non-solvent for the micronization process is able to absorb water in the range of 2-100% w/w. At temperatures in the range of 20 to 25° C., n-butanol will absorb about 9% water, iso-butanol about 8%, 1-pentanol about 2.5%, 2-pentanol about 16%, 3-pentanol about 5%, isopentanol about 2%, and tert-pentanol (tert-amyl alcohol) will absorb about 12%. (Data from the Merck Index, 11^{th} Ed.) Hence, at a dilution ratio of 50 ml of alcohol per ml of water, all of these alcohols are miscible with water. At higher temperatures, even more water can be absorbed. For the more absorbing alcohols, lower dilution ratios are possible.

Mixtures of alcohols are also potentially useful, although less preferred. Some other non-aqueous liquids may also be used as non-solvents, particularly in combination with lower alcohols; these liquids include glycols, in particular. However, single-component liquids are preferred for economy in production, and alcohols are the preferred non-solvents.

B. Stabilizers

The stability of proteins varies, and some proteins appear to benefit from being stabilized before precipitation. Other proteins, and most nucleic acids, do not require stabilization. When required, the biologically active agents in the aqueous solutions are stabilized by the addition of stabilizers to the solution. Suitable stabilizers include salts, buffers, sugars, polyols, polyalkylene glycols, polyvinylpyrrolidone, and water-soluble polymers. The function of stabilizers of this sort is the preservation of biological activity during the precipitation. The stabilizers may remain with the precipitated particles (as Zn ion does in some of the examples described herein), or may be removed from the particles by the non-solvent, or by washing. Other stabilizers will preserve the biological activity during storage, and may be added at the precipitation stage, or, often with greater economy, in the later stages of preparation. A wide variety of such materials are well-known in the art of formulation. For example antioxidants are frequently used to improve shelf life. In the preferred embodiment the stabilizing agents are mannitol and sucrose.

C. Additives

Precipitating agents may also be added to the aqueous solution. The biologically active agents can be made slightly insoluble through the addition of one or more precipitating agents. Then a precipitation reversing agent can be added to bring the biologically active agent back into the solution. Examples of suitable precipitating agents include salts, pH changes, temperature changes, polyols, polyalkylene glycols, polyvinylpyrrolidone, and water soluble polymers. Reversing agents include, depending on the method of precipitation, pH changes, dilution and chelation. The precipitating agent may be the same as or different from the stabilizing agent. The agent to be used depends on the particular biologically active agent, and typically must be determined empirically, or from known properties of the particular biologically active agent.

D. Encapsulation

In one embodiment, the micronization process is followed by additional processing in which the micronized particles of biologically active agent are microencapsulated in one or more polymers, for example, using standard microencapsulation and nanoencapsulation techniques. The micronized particles of biologically active agent, formed by precipitation in alcohol, can serve as a core material in many standard encapsulation processes. The core material typically is encapsulated in a polymeric material. Common microencapsulation techniques include interfacial polycondensation, spray drying, hot melt microencapsulation, and phase separation techniques (solvent removal and solvent evaporation). The selection of an encapsulation technique depends on the material to be encapsulated, and the therapy to be accomplished with it. Potentially suitable techniques include the following:

1. Interfacial Polycondensation

Interfacial polycondensation can be used to microencapsulate a core material in the following manner. One monomer is dissolved in a first solvent, and the core material is dissolved or suspended in the first solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

2. Spray Drying

Spray drying is typically a process for preparing 1 to 10 μm-sized microspheres in which the core material to be encapsulated is dispersed or dissolved in a polymer solution (typically aqueous), the solution or dispersion is pumped through a micronizing nozzle driven by a flow of compressed gas, and the resulting aerosol is suspended in a heated cyclone of air, allowing the solvent to evaporate from the microdroplets. The solidified particles pass into a second chamber and are collected.

3. Hot Melt Microencapsulation

Hot melt microencapsulation is a method in which a core material is added to molten polymer. This mixture is suspended as molten droplets in a nonsolvent for the polymer (often oil-based) which has been heated approximately 10° C. above the melting point of the polymer. The emulsion is maintained through vigorous stirring while the nonsolvent bath is quickly cooled below the glass transition of the polymer, causing the molten droplets to solidify and entrap the core material. Microspheres produced by this technique typically range in size from 50 μm to 2 mm in diameter. This process generally requires the use of polymers with fairly low melting temperatures (e.g., less than about 150° C., to prevent biologically active agent denaturation; preferably less than about 80° C. for most proteins and some nucleic acids), and with glass transition temperatures above room temperature, and core materials which are thermo-stable.

4. Solvent Evaporation Microencapsulation

In solvent evaporation microencapsulation, the polymer is typically dissolved in a water-immiscible organic solvent and the material to be encapsulated is added to the polymer solution as a suspension or solution in organic solvent. An emulsion is formed by adding this suspension or solution to a beaker of vigorously stirring water (often containing a surface active agent to stabilize the emulsion). The organic solvent is evaporated while continuing to stir. Evaporation results in precipitation of the polymer, forming solid microcapsules containing core material.

5. Phase Separation Microencapsulation

Phase separation microencapsulation is typically performed by dispersing the material to be encapsulated in a polymer solution by stirring. While continuing to uniformly suspend the material through stirring, a nonsolvent for the polymer is slowly added to the solution to decrease the polymer's solubility. The polymer either precipitates or phase separates into a polymer rich and a polymer poor phase, depending on the solubility of the polymer in the solvent and nonsolvent. Under proper conditions, the polymer in the polymer rich phase will migrate to the interface with the continuous phase, encapsulating the core material in a droplet with an outer polymer shell.

One embodiment of the process is described in U.S. Pat. No. 5,407,609 to Tice, et al., which discloses a phase separation microencapsulation process which reportedly proceeds very rapidly. In the method, a polymer is dissolved in a solvent, and then an agent to be encapsulated is dissolved or dispersed in that solvent. Then the mixture is combined with an excess of nonsolvent and is emulsified and stabilized, whereby the polymer solvent no longer is the continuous phase. Aggressive emulsification conditions are applied to produce microdroplets of the polymer solvent. The stable emulsion then is introduced into a large volume of nonsolvent to extract the polymer solvent and form microparticles. The size of the microparticles is determined by the size of the microdroplets of polymer solvent.

6. Phase Inversion Nanoencapsulation (PIN)

PIN is a nanoencapsulation technique which takes advantage of the immiscibility of dilute polymer solutions in select “non-solvents” in which the polymer solvent has good miscibility. The result is spontaneous formation of nanospheres (less than 1 μm) and microspheres (1-10 μm) within a narrow size range, depending on the concentration of the initial polymer solution, the molecular weight of the polymer, selection of the appropriate solvent-non-solvent pair and the ratio of solvent to non-solvent (see U.S. Pat. Nos. 6,677,869; 6,235,224; and 6,143,211 to Mathiowitz et al.). Encapsulation efficiencies are typically 75-90% and recoveries are 70-90% and bioactivity is generally well-maintained for sensitive bioagents.

“Phase inversion” of polymer solutions under certain conditions can bring about the spontaneous formation of discreet microparticles. The process, called “phase inversion nanoencapsulation” or “PIN”, differs from existing methods of encapsulation in that it is essentially a one-step process, is nearly instantaneous, and does not require emulsification of the solvent. Under proper conditions, low viscosity polymer solutions can be forced to phase invert into fragmented spherical polymer particles when added to appropriate nonsolvents.

Phase inversion phenomenon has been applied to produce macro- and micro-porous polymer membranes and hollow fibers, the formation of which depends upon the mechanism of microphase separation. A prevalent theory of microphase separation is based upon the belief that “primary” particles form of about 50 nm diameter, as the initial precipitation event resulting from solvent removal. As the process continues, primary particles are believed to collide and coalesce forming “secondary” particles with dimensions of approximately 200 nm, which eventually join with other particles to form the polymer matrix. An alternative theory, “nucleation and growth”, is based upon the notion that a polymer precipitates around a core micellar structure (in contrast to coalescence of primary particles).

The process results in a very uniform size distribution of small particles forming at lower polymer concentrations without coalescing supports the nucleation and growth theory, while not excluding coalescence at higher polymer concentrations (e.g., greater than 10% weight per volume) where larger particles and even aggregates can be formed. (Solvent would be extracted more slowly from larger particles, so that random collisions of the partially-solvated spheres would result in coalescence and, ultimately, formation of fibrous networks.) By adjusting polymer concentration, polymer molecular weight, viscosity, miscibility, and solvent:nonsolvent volume ratios, the interfibrillar interconnections characteristic of membranes using phase inversion are avoided, with the result being that microparticles are spontaneously formed. These parameters are interrelated and the adjustment of one will influence the absolute value permitted for another.

In the preferred processing method, a mixture is formed of the agent to be encapsulated, a polymer and a solvent for the polymer. The agent to be encapsulated may be in liquid or solid form. It may be dissolved in the solvent or dispersed in the solvent. The agent thus may be contained in microdroplets dispersed in the solvent or may be dispersed as solid microparticles in the solvent. The phase inversion process thus can be used to encapsulate a wide variety of agents by including them in either micronized solid form or else emulsified liquid form in the polymer solution.

The loading range for the agent within the microparticles is between 0.01-80% (agent weight/polymer weight). When working with nanospheres, an optimal range is 0.1-5% (weight/weight).

The number average molecular weight range for the polymer is on the between approximately lkDa and 150,000 kDa, and is preferably between 2 kDa and 50 kDa. The polymer concentration is typically between 0.01 and 50% (weight/volume). However, other concentration ranges may be suitable, depending primarily upon the molecular weight of the polymer and the resulting viscosity of the polymer solution. In general, the low molecular weight polymers permit usage of a higher concentration of polymer. The preferred concentration range is between approximately 0.1% and 10% (weight/volume), and is preferably below 5% (weight/volume). Polymer concentrations ranging from 1 to 5% (weight/volume) are particularly useful.

The viscosity of the polymer solution preferably is less than 3.5 cP and more preferably less than 2 cP, although higher viscosities such as 4 or even 6 cP are possible depending upon adjustment of other parameters such as molecular weight of the polymer. It will be appreciated by those of ordinary skill in the art that polymer concentration, polymer molecular weight and viscosity are interrelated, and that varying one will likely affect the others.

The nonsolvent, or extraction medium, is selected based upon its miscibility in the solvent. Thus, the solvent and nonsolvent are thought of as “pairs”. The solubility parameter (δ(cal/cm³)^{1/2}) is a useful indicator of the suitability of the solvent/nonsolvent pairs. The solubility parameter is an effective protector of the miscibility of two solvents and, generally, higher values indicate a more hydrophilic liquid while lower values represent a more hydrophobic liquid (e.g:, δi water=23.4(cal/cm³)^{1/2} whereas δi hexane=7.3 (cal/cm³)^{1/2}). Solvent/nonsolvent pairs are useful the absolute value of the difference between the δ of the solvent and the δ of the nonsolvent is less than about 6 (cal/cm³)^{1/2}. Although not wishing to be bound by any theory, an interpretation of this finding is that miscibility of the solvent and the nonsolvent is important for formation of precipitation nuclei which ultimately serve as foci for particle growth. If the polymer solution is totally immiscible in the nonsolvent, then solvent extraction does not occur and nanoparticles are not formed. An intermediate case would involve a solvent/nonsolvent pair with slight miscibility, in which the rate of solvent removal would not be quick enough to form discreet microparticles, resulting in aggregation of coalescence of the particles.

Nanoparticles generated using “hydrophilic” solvent/nonsolvent pairs (e.g., a polymer dissolved in methylene chloride with ethanol as the nonsolvent) yielded particles in the size range of 100-500 nm compared to the larger particles measuring 400-2,000 nm produced when “hydrophobic” solvent/nonsolvent pairs were used (e.g., the same polymer dissolved in methylene chloride with hexane as the nonsolvent).

Similarly, the solvent:nonsolvent volume ratio is important in determining whether microparticles would be formed without particle aggregation or coalescence. A suitable working range for solvent:nonsolvent volume ratio is from 1:40 to 1:1,000,000 (volume per volume). Preferably the working range for the volume ratios for solvent:nonsolvent is from 1:50 to 1:200 (volume per volume). Ratios of less than approximately 1:40 resulted in particle coalescence. This result may be due to incomplete solvent extraction or a slower rate of solvent diffusion into the bulk nonsolvent phase.

It will be understood by those of ordinary skill in the art that the ranges given above are not absolute, but instead are interrelated. For example, although it is believed that the solvent:nonsolvent minimum volume ratio is on the order of 1:40, it is possible that microparticles still might be formed at lower ratios such as 1:30, if the polymer concentration is extremely low, the viscosity of the polymer solution is extremely low and the miscibility of the solvent and nonsolvent is high. Thus, the polymer is dissolved in an effective amount of solvent, and the mixture of biologically active agent, polymer and polymer solvent is introduced into an effective amount of a nonsolvent, to produce polymer concentrations, viscosities and solvent:nonsolvent volume ratios that cause the spontaneous and virtually instantaneous formation of microparticles.

A variety of polymers may be used, including polyesters such as poly(lactic acid), poly(lactide-co-glycolide) in molar ratios of 50:50 and 75:25; polycaprolactone; polyanhydrides such as poly(fumaric-co-sebacic) acid or P(FA:SA) in molar ratios of 20:80 and 50:50; poly(carboxyphenoxypropane-co-sebacic) acid or P(CPP:SA) in molar ratio of 20:80; and polystyrenes (PS). Poly(ortho)esters, blends and copolymers of these polymers can also be used, as well as other biodegradable polymers and non-biodegradable polymers such as ethylenevinyl acetate and polyacrylamides.

Nanospheres and microspheres having sizes ranging from 10 nm to 10 μm have been produced by these methods. Using initial polymer concentrations in the range of 1-2% (weight/volume) and solution viscosities of 1-2 cP, with a “good” solvent, such as methylene chloride and a strong non-solvent, such as petroleum ether or hexane, in an optimal 1:100 volume ratio, generates particles with sizes ranging from 100-500 nm. Under similar conditions, initial polymer concentrations of 2-5% (weight/volume) and solution viscosities of 2-3 cP typically produce particles with sizes of 500-3,000 nm. Using very low molecular weight polymers (less than 5 kDa), the viscosity of the initial solution may be low enough to enable the use of higher than 10% (weight/volume) initial polymer concentrations which generally result in microspheres with sizes ranging from 1-10 μm. In general, it is likely that with concentrations of 15% (weight/volume) and solution viscosities greater than about 3.5 cP, discreet microspheres will not form but, instead, will irreversibly coalesce into intricate, interconnecting fibrillar networks with micron thickness dimensions.

These encapsulation methods can result in product yields greater than 80% and encapsulation efficiencies as high as 100%, of nano- to micro-sized particles.

The methods described herein also can produce microparticles and nanoparticles characterized by a homogeneous size distribution. The methods described herein can produce, for example, nanometer sized particles which are relatively monodisperse in size. By producing a microparticle that has a well-defined and less variable size, the properties of the microparticle such as when used for release of a biologically active agent can be better controlled. Thus, the methods permit improvements in the preparation of sustained release formulations for administration to subjects.

The methods are also useful for controlling the size of the microspheres. This is particularly useful where the material to be encapsulated must first be dispersed in the solvent and where it would be undesirable to sonicate the material to be encapsulated. The mixture of the material to be encapsulated and the solvent (with dissolved polymer) can be frozen in liquid nitrogen and then lyophilized to disperse the material to be encapsulated in the polymer. The resulting mixture then can be redissolved in the solvent, and then dispersed by adding the mixture to the nonsolvent. This methodology was employed in connection with dispersing DNA (see WO 01/51032 to Brown University Research Foundation).

In many cases, the methods can be carried out in less than five minutes in the entirety. Preparation time may take anywhere from one minute to several hours, depending on the solubility of the polymer and the chosen solvent, whether the agent will be dissolved or dispersed in the solvent and so on. Nonetheless, the actual encapsulation time typically is less than thirty seconds.

After formation of the microcapsules, they are collected by centrifugation, filtration, or other standard techniques. Filtering and drying may take several minutes to an hour depending on the quantity of material encapsulated and the methods used for drying the nonsolvent. The process in its entirety may be discontinuous or a continuous process.

III. Uses for Micron and Submicron Particles

The biologically active agent particles may be delivered to patients for the treatment of diseases and disorders. In one embodiment, the particles are suitable for delivery to mucosal surfaces, such as in intranasal, pulmonary, vaginal, or oral administration. In another embodiment, the particles are suitable for parenteral administration. The particles will not clog blood vessels when administered parenterally due to their small size.

In a preferred embodiment, the biologically active agent is insulin. In the most preferred embodiment, the insulin particles are coated with a bioadhesive polymer, such as a polyanhydride, to improve their uptake from the intestine.

Additionally, protein particles and other biologically active agent particles formed in this manner can be used as aggregates in larger capsules. The small particle size with a suitable coating improves delivery across the intestine, leading to clinically useful bio-availabilities. Additionally, these small biologically active agent particles can be used for immunization, optionally in admixture with immune system stimulants and adjuvants. This can involve “Peyer's patches” and similar organs, in the intestine and in other mucosae. Nucleic acid particles can be used to transform cells and to engage in other intracellular uses of nucleic acids, of which a large variety have been proposed in the art, e.g. (plasmids and RNA silencing). In general, the particles of biologically active agents are advantageous for use in the known therapeutic uses for the particular biologically active agent.

It is well-known to those skilled in the art that micronized drug particles may be administered to patients using a full range of routes of administration. As an example, micronized drug particles may be blended with direct compression or wet compression tableting excipients using standard formulation methods. The resulting granulated masses may then be compressed in molds or dies to form tablets and subsequently administered via the oral route of administration. Alternately micronized drug granulates may be extruded, spheronized and administered orally as the contents of capsules and caplets. Tablets, capsules and caplets may be film coated to alter dissolution of the delivery system (enteric coating) or target delivery of the microspheres to different regions of the gastrointestinal tract. Additionally, micronized drug may be orally administered as suspensions in aqueous fluids or sugar solutions (syrups) or hydroalcoholic solutions (elixirs) or oils. In the preferred embodiment the particles are suitable for oral administration.

Micronized drug may be co-mixed with gums and viscous fluids and applied topically for purposes of buccal, rectal or vaginal administration. Micronized drug may also be co-mixed with gels and ointments for purposes of topical administration to epidermis for transdermal delivery.

Micronized drug may also be suspended in non-viscous fluids and nebulized or atomized for administration of the dosage form to nasal membranes Micronized drug may also be delivered parenterally by either intravenous, subcutaneous, intramuscular, intrathecal, intravitreal or intradermal routes as sterile suspensions in isotonic fluids.

Finally, micronized drug may be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., air, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the microparticle (see, for example, Sciarra and Cutie, “Aerosols,” in Remington's Pharmaceutical Sciences, 18th ed., p. 1694-1712 (1990)).

Micronized drug particles, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The methods and compositions described herein will be further understood by reference to the following non-limiting examples.

### EXAMPLES

Examples 1-10 describe different micronized insulin formulations and methods for making these formulations. The gross yield is usually not corrected for the presence of non-insulin material in the precipitate. In Example 5 below, approximately 30% of the recovered weight was not insulin, but buffers, salts, etc.

### Example 1

### Preparation of Tert Butanol (tBA) Insulin Particles by Ultrasonic Nozzle

0.25 gm of zinc insulin (Gibco Cat #18125-039, Lot 1108537) was dissolved in 25 ml of 0.01N HCl. 0.80 ml of 10% zinc sulfate solution (w/v) was slowly added with shaking to barely precipitate the insulin from the solution. 3.0 ml of 0.01N HCl was added to back-titrate and redissolve the insulin. Total volume was 28.8 ml (“balanced zinc insulin solution”).

23.8 ml of balanced zinc insulin solution was drawn into a 20 ml glass syringe and the solution was delivered to an ultrasonic nozzle (Sonotek, Cat#12354) at a rate of 0.6 ml/min using a syringe pump (Harvard Apparatus syringe pump, model 55-4140). The ultrasonic nozzle was set 1 cm from the surface and 1 cm from the center of a 500 mL bath of tBA (JT Baker Lot t15B09) in a 600 ml glass beaker. The power output of the nozzle was 2.5W. The bath was stirred at 400 rpm and the temperature was maintained at 29° C. After 40 min, the particles suspended in tBA were decanted into 2×250 ml amber polyethylene (PE) bottles, flash frozen by immersion in liquid nitrogen for 5 min and lyophilized for 5 days.

The gross yield of tBA insulin particles was 70% of the starting weight. The particles were in the form of a fine, white powder and had a bulk density of ˜0.1 gm/ml. Scanning electron microscope (SEM) analysis indicated discrete particles with diameters in the range of about 300-600 nm.

### Example 2

### Preparation of tBA Insulin Particles by Coacervation

0.25 gm of zinc insulin (Gibco Cat #18125-039, Lot 1108537) was dissolved in 25 ml of 0.01N HCl. 0.80 ml of 10% zinc sulfate solution (w/v) was slowly added with shaking to barely precipitate insulin from solution. 3.0 ml of 0.01N HCl was added to back-titrate and redissolve the insulin. The total volume of the balanced zinc insulin solution was 28.8 ml.

5.0 ml of balanced zinc insulin solution was transferred to a 125 ml Wheaton glass bottle and 110 ml of tBA (JT Baker Lot t15B09), maintained at 29° C. was slowly added over the course of 10 sec. The mixture was capped and mixed by inversion once. The contents were transferred to 4×50 ml conical centrifuge tubes, centrifuged at 6K rpm for 20 min in an IEC centrifuge (IEC model CL2). The supernatant fluids were discarded and the pellets were flash frozen by immersion in liquid nitrogen for 5 min and lyophilized for 5 days.

The gross weight of tBA insulin particles was 100% of the starting weight. The particles were in the form of a white powder and had a bulk density of ˜1 gm/ml. SEM analysis indicated discrete particles measuring 300-600 nm.

### Example 3

### Preparation of tBA Insulin Particles by Precipitation

0.25 gm of zinc insulin (Gibco Cat # 18125-039, Lot 1108537) was dissolved in 25 ml of 0.01N HCl. 0.80 ml of 10% zinc sulfate solution (w/v) was slowly added with shaking to barely precipitate insulin from solution. 3.0 ml of 0.01N HCl was added to back-titrate and redissolve the insulin. The total volume of the balanced zinc insulin solution was 28.8 ml.

28.8 ml of balanced solution was quickly dispersed into 720 ml of tBA (25×the volume of balanced solution), maintained at 29° C., in a 3.5L S/S pressure pot. The pot was sealed and mixed by swirling for 10 sec. The contents were filtered with 0.22 μm Teflon filter (Osmonics F02LP0925) in a 9 cm S/S filter holder, at a positive nitrogen pressure of 20 psi. The retentate was removed from the filter by scraping with a spatula, transferred to a clean, tared scintillation vial and flash frozen by immersion in liquid nitrogen for 5 min. The tBA particles were lyophilized for 2 days.

The gross yield of tBA insulin particles was 80%. The particles were in the form of a white powder and had a bulk density of ˜1 gm/ml. SEM analysis indicated discrete particles measuring 300-600 nm.

Discussion

Examples 1, 2 and 3 describe three different methods for micronizing insulin through the use of tBA. All of these methods were effective at forming small (300-600 nm), fine particles of insulin.

### Example 4

### Preparation of tBA Insulin Particles by Precipitation

5 gm of zinc insulin (Spectrum Lot RI0049) was dissolved in 500 ml of 0.01N HCl. 32.0 ml of 10% zinc sulfate solution was slowly added with shaking to barely precipitate insulin from solution. 120.0 ml of 0.01N HCl was added to back titrate and redissolve the insulin. The total volume of the balanced zinc insulin solution was 652 ml.

652 ml of balanced solution was placed in a water bath at 28° C. for 10 min and then poured into 9780 ml of tBA (15× the volume of balanced solution), maintained at 28° C. in a 3.5 L S/S pressure pot. The pressure pot, filter holder and tubing were immersed in a water bath at 28° C. The mixture was stirred with a spatula for 10 sec and the pot was sealed and mixed by swirling for 10 sec. The contents were filtered with 0.22 μm Teflon filter (Millipore FGLP0950) in a 9 cm S/S filter holder, at a positive nitrogen pressure of 20 psi. The retentate was removed from the filter by scraping with a spatula, transferred to a clean, tared scintillation vial and flash frozen by immersion in nitrogen for 5 min. The tBA particles were lyophilized for 3 days.

The gross yield of tBA insulin particles was about 102% of the starting weight, without correction for salts, etc. The particles were in the form of a white powder and had a bulk density of ˜1 gm/ml. A portion of the retentate was resuspended in fresh tBA and flash frozen and lyophilized for 1 day. The apparent bulk density of this material was ˜0.1 gm/ml.

### Example 5

### Preparation of tBA Insulin Particles by Precipitation

8 gm of zinc insulin (Spectrum Lot RI0049) was dissolved in 800 ml of 0.01N HCl. 51.2 ml of 10% zinc sulfate solution was slowly added with shaking to barely precipitate insulin from solution. 160.0 ml of 0.01N HCl was added to back titrate and redissolve the insulin. The total volume of the balanced zinc insulin solution was 1011 ml.

1011 ml of balanced solution was placed in a water bath at 28° C. for 10 min and then poured into 15168 ml of tBA (15×the volume of balanced solution), maintained at 28° C. in a 20 L S/S pressure pot. The pressure pot, filter holder and tubing were immersed in a water bath at 28° C. The mixture was stirred with a spatula for 10 sec and the pot was sealed and mixed by swirling for 10 sec. The contents were filtered with 0.22 μm Teflon filter (Millipore FGLP0950) in a 9 cm S/S filter holder, at a positive nitrogen pressure of 20 psi. The retentate was removed from the filter by scraping with a spatula, transferred to a clean, tared plastic jar, resuspended in fresh tBA and flash frozen by immersion in nitrogen for 5 min. The tBA particles were lyophilized for 6 days. The gross yield of tBA insulin particles exceeded 100%. The particles were in the form of a fine, white powder and had a bulk density of ˜0.1 gm/ml.

### Example 6

### Composition of tBA Insulin Powders

The formulation described in Example 5 was analyzed for insulin content by HPLC, for water content by Karl Fischer titration, for zinc content by EDTA titration, for residual tBA content by gas chromatography and for sulfate content by LC/MS/MS. The amount of insulin was 69% w/w; the amount of water was 11% w/w; the amount of tBA was 1% w/w; and the amounts of zinc and sulfate were each 10% w/w. Size exclusion and reversed phase HPLC of the insulin indicated that insulin dimer was 4% w/w of the total insulin and desamido (deamidated) insulin was 2% w/w of the total insulin.

### Example 7

### Bioactivity of tBA Insulin In Vivo

A tBA insulin formulation prepared as described in Example 1 was tested for bioactivity by intraperitoneal (IP) injection into fasted rats. The dose was 1.5 IU/kg. The area under the curve (AUC) of the glucose depression curve (absolute values) over 6 hrs was compared to the AUC of IP injections of 0, 0.75, 1, 1.5, 3, and 5 IU/kg bovine zinc insulin over the same time period. Based on these results, the bioactivity of the tBA formulation was estimated at greater than 80% of the bovine zinc insulin.

### Example 8

### Phase Inversion Nano-Encapsulation of tBA Insulin in Eudragit S100/FASA

19.2 mg of tBA insulin prepared as described in Example 5 was dispersed by bath sonication in a polymer solution containing 301.9 mg of Eudragit S100 (Rohm and Haas) and 302.6 mg of poly(fumaric-co-sebacic) acid (P(FA:SA) 20:80, Spherics Inc) in 35 ml of acetone: dichloromethane: isopropanol (4:2:1, v:v:v). The mixture was dispersed into 3 L of pentane containing 6 ml of SPAN 85 (Spectrum) and collected by filtration with 0.22 μm Teflon filter (Millipore FGLP0950) in a 9 cm S/S filter holder, at a positive nitrogen pressure of 20 psi. The yield was 92.4%. The particles were analyzed for size distribution in 50 mM citrate buffer, pH 5.5 using a Coulter Multisizer III. The particle distribution was lower than the limit of detection of the instrument (typically particle size less than 1-1.5 microns).

### Example 9

### Bioactivity of tBA Insulin in Eudragit S100/FASA Nanoparticles in vivo

The tBA insulin nanoparticle formulation described in Example 8 was tested for bioactivity by IP injection into fasted rats at 1.5 IU/kg. The AUC of the glucose depression curve (absolute values) over 6 hrs was compared to the AUC of IP injections of 0, 0.75, 1, 1.5, 3, and 5 IU/kg bovine zinc insulin over the same time period. Based on these results, the IP injection bioactivity of the tBA formulation was estimated at greater than 56% of the bovine zinc insulin. The bioactivity of a tBA insulin nanoparticle formulation analogous to the formulation in Example 8, but using Eudragit S100 alone was greater than 97% of the bovine zinc insulin. Thus, the encapsulated insulin nanoparticles had a greater bioavailability than the non-encapsulated insulin nanoparticles (see Example 7).

### Example 10

### Oral Bioactivity of tBA Insulin in P(FA:SA) Nanoparticles in Vivo

A 2% tBA insulin formulation in poly(fumaric-co-sebacic) acid (P(FA:SA)) was prepared by phase inversion nanoencapsulation using pentane as non-solvent and dichlormethane as the solvent for P(FA:SA). The formulation was tested for oral bioactivity in non-fasted, diabetic rats at a dose level of 250 IU/kg. Plasma insulin was measured by ELISA and glucose depression was measured by glucometer. The oral bioavailability of insulin in this model was 6.5% compared to subcutaneous injection of insulin.

In a separate study, the bioavailability for non-encapsulated insulin was tested. Non-encapsulated insulin resulted in a bioavailability of less than 1%, which is much lower than the bioavailability for encapsulated insulin.

### Example 11

### Precipitation of tBA Growth Hormone Particles with Ultrasonic Nozzle

5 mg of human growth hormone (hGH Serono Lot PGRE9901) in 0.25 ml of phosphoric acid/sucrose solution was diluted 1:1 with 250 μL of distilled water to which 50 μl of 10% Pluronic F127 w/v was added.

The solution was delivered to an ultrasonic nozzle (Sonotek, Cat#12354) at a rate of 0.6 ml/min by gravity feed. The ultrasonic nozzle was set 1 cm from the surface and 1 cm from the center of a 5 mL volume of tBA maintained at ˜30° C. in a 20 ml glass vial. The power output of the nozzle was 1.5 W. The suspension was shell frozen by immersion in liquid Nitrogen for 5 min and lyophilized for 2 days. SEM analysis indicated discrete particles measuring 300-600 nm. HPLC analysis indicated that 89% of the hGH was in the native state with 9% undergoing aggregation.

### Example 12

### Reduced Aggregation in tBA Growth Hormone Nanoparticles

Three aliquots containing 2.7 mg of human growth hormone (hGH Serono Lot PGRE9901) in 0.27 ml of phosphoric acid/sucrose solution were prepared and to each aliquot was added 63 μL of 8% mannitol (w/v) and 25 μL of 2% PLURONIC® F127 (w/v). Aliquot #1 received no additions (control). 1 μL of 10% ZnSO₄ w/v was added to Aliquot #2. 10 μL of 10% ZnSO₄ w/v was added to Aliquot #3. Each aliquot was individually dispersed into separate 45 ml volumes of tBA at 25° C. in 50 ml conical plastic centrifuge tubes. The tBA precipitates were centrifuged for 10 min at 3 KG in an IEC clinical centrifuge and the supernatant fluids discarded. The pellets were resuspended in 0.7 ml of tBA containing 50 μL of 8% mannitol (w/v) and 20 μL of 2% PLURONIC® F127 (w/v). The suspensions were shell frozen by immersion in liquid Nitrogen for 5 min and lyophilized for 1 day. HPLC analysis indicated that the control sample in Aliquot #1 had 90% of the hGH in the native state and 10% aggregation; zinc precipitated sample in Aliquot #2 had 92% of the hGH in the native state and 8% aggregation; zinc precipitated sample in Aliquot #3 (10 times the amount of zinc added to Aliquot #2) had 99% of the hGH in the native state and 3% aggregated (within experimental error). Thus, the results indicate that zinc stabilized hGH against aggregation during the tBA micronization process.

### Example 13

### Precipitation of tBA Growth Hormone Particles and Encapsulation in PLGA by PIN

10 mg of human growth hormone (hGH Serono Lot PGRE9901) in 0.50 ml of phosphoric acid/sucrose solution was diluted 1:1 with 500 μL of distilled water to which 100 μL of 10% PLURONIC® F127 w/v was added. The solution was dispersed into 40 ml of tBA at 30° C. in a 50 ml conical plastic centrifuge tube and vortexed for 10 sec. The suspension was centrifuged at 3 KG for 10 min in an IEC Clinical centrifuge and the supernatant fluid was decanted. An aliquot of the suspension was air-dried and examined by SEM. tBA hGH particles were spherical and ranged in size from 200-500 nm.

To encapsulate the micronized hGH particles by Phase inversion nanoencapsualtion (PIN), the pellet was resuspended in 0.5 ml of supernatant fluid and 0.5 ml of ethyl acetate was added as a transition solvent. The mixture was vortexed for 10 sec and added to 3.33 ml of 3% PLGA RG502H (50:50, Boehringer Ingelheim) in dichloromethane. The suspension was vortexed for 10 sec and dispersed into 200 ml of petroleum ether. The encapsulated tBA-hGH was recovered by filtration, air-dried and then vacuum-dried for 18 hrs to remove residual solvents. 55.9 mg of PIN particles were recovered.

### Example 14

### Precipitation of Insulin with Different Alcohols

To prepare a balanced zinc insulin solution, 0.3 ml of 10% zinc sulfate w/v solution was added to 10 ml of a 10 mg/ml zinc insulin solution in 0.01 N HCl, resulting in a fine protein precipitate. 1-1.2 ml of 0.01 N HCl was added to the mixture to “back-titrate” and redissolve the insulin.

Aliquots of the balanced zinc insulin solution were used to test the effect of different alcohols on insulin precipitation. The alcohols that were tested were: ethanol, n-propanol, 1-butanol, 2-butanol, 1-pentanol, 3-pentanol, 3-methyl-1-butanol, and tert-amyl alcohol.

For each sample, 1 ml aliquots of balanced zinc insulin solution were pipetted into the bottom of 50 ml plastic conical centrifuge tubes. For each alcohol, 40 ml of the alcohol to be tested was added to an aliquot of the zinc insulin solution, and the mixture was agitated by inversion three times. The precipitate was collected by centrifugation at 3000 rpm for 30 min in a tabletop centrifuge. The supernatant alcohol solution was aspirated and discarded. The insulin precipitate was frozen in liquid nitrogen and lyophilized for two days. The morphology and size of the protein particles were evaluated by scanning electron microscopy. The results are listed in Table 1. Yield of recovered protein precipitate, based upon qualitative evaluation of the size of the centrifugal pellet, was scored on a qualitative visual scale of 1 (least) to 5 (greatest).

TABLE 1
Morphology, Size and Yield of Particles in Different Non-solvents

| | | | | Yield (Size of |
|---|---|---|---|---|
| | Primary Particle | | | Pellet) |
| Alcohol | Size | | Morphology | (1-5 + scale) |
| Ethanol | 50-200 | nm | Aggregated Plates | 1 |
| | | | (50 + microns) | |
| n-Propanol | 100-300 | nm | Aggregated Plates | 1 |
| | | | (50 + microns) | |
| 1-Butanol | 1-30 | microns | Irregular porous | 2 |
| | | | discrete particles | |
| 2-Butanol | 1-30 | microns | Irregular porous | 3 |
| | | | discrete particles | |
| 1-Pentanol | 5-50 | microns | Irregular porous | 2 |
| | | | discrete particles | |
| 3-Pentanol | 0.5-1 | microns | Irregular spherical | 5 |
| | | | particles | |
| 3-Methyl-1- | 1-30 | microns | Irregular porous | 4 |
| Butanol | | | discrete particles | |
| Tert-Amyl | 2-50 | microns | Irregular porous | 4 |
| Alcohol | | | discrete particles | |

tBA was not run in this series, but comparable values were obtained in Examples 1-5. Thus particles obtained using tBA would be 300-600 nm regular, spherical, non-aggregated particles, with a yield in the 2 to 3 region.

The aggregated plates obtained in the ethanol and n-propanol samples appeared to be aggregations of small, smooth particles, probably formed during collection of the particles.

The results indicate that t-butanol is the preferred non-solvent due to the size, shape and yield of the resulting particles. However, optimization could produce equivalent results from C1 to C3 alcohols.

### Example 15

### Micronization of hIL-12

Recombinant Human Interleukin-12 (hIL-112) was obtained from Genetics Institute and tBA from EM Science. hIL-12 (500 microliters at 2.79 mg/ml) was injected into tBA (5 ml). A fine precipitate formed immediately. The dispersion was rapidly frozen in liquid nitrogen (15 minutes) and the solvent removed by lyophilization for 48 hours. The resulting powder was visualized by SEM. The stability following micronization was assayed by SDS-PAGE (Invitrogen) and BCA (Pierce) assay of hIL-12 resolubilized in 10 mM PBS according to manufacturer's instructions.

The morphology of micronized hIL-12 was determined by SEM. The particles consisted of larger (˜2 micron) crystals, resulting from the buffer salts, and smaller (<1 micron) particles, corresponding to the hIL-12.

SDS-PAGE (denaturing, non-reducing) analysis was used to compare tBA-treated hIL-12 particles with the control stock, with and without lyophilization. The stained gels showed that some of the micronized hIL-12 was irreversibly denatured and aggregated into dimers, trimers, and aggregates too large to enter the gel, even in the presence of SDS. The process of lyophilization itself did not produce irreversible aggregation. This was confirmed by bichloroacetic acid (BCA) analysis of protein concentration of hIL-12 redissolved after lyophilization alone, or after micronization followed by lyophilization. The lyophilized-only protein was 99% soluble, while the micronized protein was 71% soluble. Thus a disagreggating agent should be included to prevent aggregation when micronizing hIL-12 using tBA.

### Example 16

### Micronization of Ricin Toxoid

Ricin toxoid (RT) was obtained from a collaborator and tBA was purchased from EM Science. RT (100 microliters at 5 mg/ml) was injected into tBA (1 ml). A fine precipitate formed immediately. The dispersion was rapidly frozen in liquid nitrogen (15 minutes) and the solvent removed by lyophilization for 48 hours. The resulting powder was visualized by SEM. The stability following micronization was assayed by SDS-PAGE (INVITROGEN®) of RT resolubilized in 10 mM PBS according to manufacturer's instructions.

SEM showed smaller particles (less than 1 micron) corresponding to ricin toxoid, and larger (˜2 micron) crystals corresponding to the buffer salt. SDS-PAGE showed a closely similar pattern of aggregation in both starting RT and in micronized RT; no clear increase in aggregation was observed in the micronized RT.

### Example 17

### Micronization of RNA

Yeast RNA was purchased from AMBION® and tBA was purchased from EM Science. RNA (100 microliters at 10 mg/ml) was injected into tBA (1 ml). A fine precipitate formed immediately. The dispersion was rapidly frozen in liquid nitrogen (15 minutes) and the solvent removed by lyophilization for 48 hours. The resulting powder was visualized by SEM. The stability following micronization was assayed by agarose gel electrophoresis of RNA resolubilized in 10 mM PBS according to manufacturer's instructions.

The morphology of the micronized RNA by SEM showed particles with a submicron size distribution. Agarose gel electrophoresis followed by ethidium bromide staining showed no difference between the micronized and control RNA in apparent molecular size.

### Example 18

### tBA Micronization of an Antibody

Rabbit gamma globulin was obtained from Pierce and tBA was from EM Science. The antibody (100 microliters at 10.6 mg/ml) was injected into tBA (1 ml). A fine precipitate formed immediately. The dispersion was rapidly frozen in liquid nitrogen (15 minutes) and the solvent removed by lyophilization for 48 hours. The resulting powder was visualized by SEM. Particles appeared to be 0.5 to 2 microns in diameter.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

1. A method for making micronized biologically active agents comprising
dissolving a biologically active agent in an aqueous solution,
mixing a nonsolvent with the aqueous solution, wherein the nonsolvent is a C1 to C6 alcohol or mixture thereof that absorbs water in the range of 2-100% w/w, and
precipitating particles of the biologically active agents out of the nonsolvent:aqueous solution combination to produce particles having diameters in the range of about 100 to 2000 nm.

2. The method of claim 1, wherein the aqueous solution further comprises one or more stabilizers selected from the group consisting of salts, buffers and water soluble polymers.

3. The method of claim 2, wherein the stabilizer is a water soluble polymer selected from the group consisting of polyols, polyalkylene glycols, and polyvinylpyrrolidone.

4. The method of claim 1, wherein the nonsolvent is an aliphatic C1 to C6 alcohol, or a mixture thereof.

5. The method of claim 4, wherein the nonsolvent is an aliphatic C2 to C5 alcohol, or a mixture thereof.

6. The method of claim 5, wherein the nonsolvent is tertiary butyl alcohol.

7. The method of claim 1, wherein the biologically active agent is selected from the group consisting of proteins, peptides, and nucleic acids.

8. The method of claim 7, wherein the biologically active agent is insulin.

9. The method of claim 7, wherein the aqueous solution further comprises a disaggregating agent.

10. The method of claim 1, further comprising encapsulating the biologically active agents in a polymer.

11. The method of claim 10, wherein the polymer is selected from the group consisting of poly(hydroxy acids), polyanhydrides, polyorthoesters, polyamides, polycarbonates, polyalkylenes, polyalkylene glycols, polyalkylene oxides, poly(meth) acrylic acids and their lower alkyl esters, polyvinyl alcohols, and copolymers and mixtures thereof.

12. A composition comprising a biologically active agent in the form of particles having an average diameter of 200 nm to 600 nm, wherein the biologically active agent is selected from the group consisting of proteins, peptides, nucleic acids, and hydrophilic, synthetic molecules, and the particles are formed by dissolving a biologically active agent in an aqueous solution, mixing a nonsolvent with the aqueous solution, wherein the nonsolvent is a C1 to C6 alcohol or mixture thereof that absorbs water in the range of 2-100% w/w, and precipitating particles of the biologically active agents out of the nonsolvent:aqueous solution.

13. The composition of claim 13, wherein the biologically active agent is insulin.

14. The composition of claim 12, wherein the particles are encapsulated in a polymer.

15. The composition of claim 14, wherein the polymer is selected from the group consisting of poly(hydroxy acids), polyanhydrides, polyorthoesters, polyamides, polycarbonates, polyalkylenes, polyalkylene glycols, polyalkylene oxides, poly(meth) acrylic acids and their lower alkyl esters, polyvinyl alcohols, and copolymers and mixtures thereof.

16. A method for treating a disease or disorder comprising administering to a patient particles of biologically active agents having an average diameter of 100 nm to 2000 nm, wherein the biologically active agent is selected from the group consisting of proteins, peptides and nucleic acids, and the particles are formed by dissolving a biologically active agent in an aqueous solution, mixing a nonsolvent with the aqueous solution, wherein the nonsolvent is a C1 to C6 alcohol or mixture thereof that absorbs water in the range of 2-100% w/w, and precipitating particles of the biologically active agents out of the nonsolvent:aqueous solution

17. The method of claim 16, wherein the biologically active agent is encapsulated in a polymer.

18. The method of claim 17, wherein the polymer is selected from the group consisting of poly(hydroxy acids), polyanhydrides, polyorthoesters, polyamides, polycarbonates, polyalkylenes, polyalkylene glycols, polyalkylene oxides, poly(meth) acrylic acids and their lower alkyl esters, polyvinyl alcohols, and copolymers and mixtures thereof.

19. The method of claim 16, wherein the particles are administered to a mucosal surface.

20. The method of claim 19, wherein the particles are administered orally or by inhalation.

21. The method of claim 16, wherein the biologically active agent is selected from the group consisting of insulin, human growth hormone, enzymes, and RNA.
